(19) United States
(12) Patent Application Publication
(10) Pub. No.: US 2022/0305058 A1
(43) Pub. Date: Sep. 29, 2022

(54) MESENCHYMAL STROMAL CELL BONE GRAFT MATERIAL

(71) Applicant: Johann Wolfgang Goethe-Universität Frankfurt am Main, Frankfurt am Main (DE)

(72) Inventors: Dirk HENRICH, Frankfurt am Main (DE); Ingo Marzi, Königstein (DE); Peter Bader, Dreieich (DE); Selim Kuci, Frankfurt am Main (DE); Zyrafete Kuci, Frankfurt am Main (DE); Thomas Klingebiel, Frankfurt am Main (DE); Halvard Bönig, Frankfurt (DE); Erhard Seifried, Frankfurt am Main (DE)

(21) Appl. No.: 17/635,664

(22) PCT Filed: Aug. 17, 2020
(86) PCT No.: PCT/EP2020/073012

§ 371 Date: Feb. 15, 2022

(30) Foreign Application Priority Data
Aug. 16, 2019 (EP) 19192111.3

(51) Int. Cl. A61K 35/32; A61L 27/38; A61L 27/56; A61L 27/46

(52) U.S. Cl. CPC A61K 35/32; A61L 27/3834; A61L 27/56; A61L 27/46; A61L 2430/02

(57) ABSTRACT

The invention pertains to the use of mesenchymal stromal cells (MSC) in the treatment of bone disorders or injuries. The invention provides MSC and preparations of specifically pooled MSC for use in the manufacturing of bone graft material for implanting into or attaching to bones in order to enhance bone regeneration after surgery or injury, or to treat various bone disorders, such as osteonecrosis. The invention provides bone graft material, a method for its production, bone graft implants, and medical methods and uses of the inventive products.

### FIELD OF THE INVENTION

The invention pertains to the use of mesenchymal stromal cells (MSC) in the treatment of bone disorders or injuries. The invention provides MSC and preparations of specifically pooled MSC for use in the manufacturing of bone graft material for implanting into or attaching to bones in order to enhance bone regeneration after surgery or injury, or to treat various bone disorders, such as osteonecrosis. The invention provides bone graft material, a method for its production, bone graft implants, and medical methods and uses of the inventive products.

### DESCRIPTION

Bone formation and degradation are tightly regulated by growth factor signaling between osteoblasts that are responsible for bone formation and osteoclasts that are responsible for bone re-absorption. Coupling bone formation by osteoblasts with degradation by osteoclasts has recently become a topic of intense study; with the list of growth factors identified as coupling factors expanding. Coupling bone formation with bone re-absorption requires the recruitment of osteoblasts and osteoclasts in parallel with the recruitment of their respective progenitor cells. Osteoblasts derive from mesenchymal stem cell (MSC) while osteoclasts derive from monocytes that are a part of the myeloid-lineage; however, it remains unknown how MSC or monocytes migrate from their niche in the bone marrow to sites of new bone formation. The current understanding of the spatial and temporal regulation of osteogenesis proposes that MSC migrate from their bone marrow niche to the endosteal surface; where the MSC differentiate into osteoblasts that produce new bone. In parallel, monocytes also migrate from their bone marrow niche to the endosteal surface; where they subsequently differentiate into osteoclasts that re-absorb bone. Growth factors known to regulate bone formation include TGFβ-, BMP- and the canonical Wnt-ligands. Osteoclast formation from monocyte precursors and bone re-absorption are regulated through the expression of MSCF, OPG and RANK-ligand. In parallel, osteoclast activity is also regulated by the expression of the TGFβ-, BMP- and the non-canonical Wnt-ligands. However, many developmental growth factors involved in tissue patterning, including TGFβ-, BMP- and the Wnt-ligands, promote bone formation and re-absorption. The maintenance of healthy bone requires constant remodeling, in which bone is made and destroyed continuously.

The introduction of an implant into bone results in a biochemical cascade that drives the pro-inflammatory response that is partially mediated by macrophage activity, which are derived from the myeloid lineage and can contribute to the degradation of bone or an implant material. Currently implants and implant materials are chosen to minimize the macrophage response while being optimally osteo-conductive and promoting maximum bone-implant integration. Alternatively, the introduction of autograft with an implant or the use of devitalized bone tissue graft (autograft) has been employed in concert with the material properties of an implant as a means of increasing osteo-integration; however, these approaches have often been problematic. Ideally, materials could be designed to be both self-organizing and self-assembling.

MSCs are stromal cells that are plastic-adherent and able to differentiate into osteoblasts, chondroblasts, and adipocytes. They express biomarkers including CD73, CD90, and CD105, and they must not express CD14 or CD11b, CD34. MSC can be derived mainly from bone marrow, but also umbilical cord blood or from other sources. In particular bone marrow derived MSC have been most extensively studied both in preclinical and clinical experiments: the effects of such MSC in fracture healing, spinal fusion, and osteonecrosis have been demonstrated (Su P, et al, Int J Mol Sci. 2018 Aug. 9; 19(8)).

The clinical potential of MSC in the treatment of bone diseases such as osteonecrosis was shown in Mueller et al (Leukemia (2008) 22, 2054-2061). A three week in vitro expansion of autologous MSC was used in patients suffering from steroid induced osteonecrosis. The treatment was safe and resulted in clinical improvement of patients.

Goomez-Barrena et al, (Biomaterials 196 (2019) 100-108) show a successful European, multicentric, first in human clinical trial using calcium phosphate bioceramic granules associated during surgery with autologous mesenchymal stromal cells expanded from bone marrow (BM-hMSC) under good manufacturing practices, in patients with long bone pseudarthrosis. In this study, in vitro expanded MSC are combined with the bone graft material during surgery and implanted into the bone.

However, still a limiting factor for the use of MSC in the treatment of bone disorders and injury is the long lasting in vitro preparation of autologous MSC. Autologous bone marrow samples are obtained from a patient, MSCs are in vitro isolated therefrom and expanded to obtain sufficient cells for engraftment. The procedure takes weeks in order to obtain MSC preparations usable for implantation. Hence, it was an object of the present invention to develop an “off-the shelf” MSC functionalized bone graft material.

### BRIEF DESCRIPTION OF THE INVENTION

Generally, and by way of brief description, the main aspects of the present invention can be described as follows:

In a first aspect, the invention pertains to bone graft material for use in the treatment of a subject, comprising a scaffold material and a biological cell material, wherein the biological cell material comprises cell material genetically allogenic to the subject.

In a second aspect, the invention pertains to bone graft implant, comprising a scaffold material and a biological cell material, wherein the bone graft implant is in the form of an implantable tablet having a height of 1 to 10 mm, preferably 3 to 5 mm, and/or a diameter of 1 to 15 mm, preferably 5 to 8 mm.

In a third aspect, the invention pertains to a method for producing cryopreserved bone graft material, comprising the steps of
Seeding biological cell material as recited in any one of the aspects of the invention on a scaffold material as recited in any of the aspects of the invention to obtain a bone graft material, and
Immediately cryopreserving the bone graft material obtained in the first step.

In a fourth aspect, the invention pertains to a method of producing a bone graft implant, the method comprising the step of compressing a bone graft material recited in any one of the preceding claims into a tablet, preferably having a height of 1 to 10 mm, preferably 3 to 5 mm, and/or a diameter of 1 to 15 mm, preferably 5 to 8 mm.

In a fifth aspect, the invention pertains to a therapeutic kit, comprising
A first container comprising a scaffold material recited in any one of the preceding aspects,
A second container comprising a biological cell material recited in any one of the preceding aspects.

In a sixth aspect, the invention pertains to a mesenchymal stromal cell (MSC), or a MSC preparation for use in the treatment of a disease, wherein the treatment comprises the administration of the MSC, or the MSC preparation, to a subject in need of the treatment by adhering the MSC or MSC preparation to a bone graft material to obtain an MSC-bone graft material, and implanting or attaching the MSC-bone graft material into/to one or more bone(s) of the patient.

In a seventh aspect, the invention pertains to a use of a MSC, or a MSC preparation, in the manufacture of a medicament, preferably a bone graft material, for use in the treatment of a bone injury or disorder.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to bone graft material, comprising a scaffold material and a biological cell material, wherein the biological cell material comprises cell material genetically allogenic to the subject to be treated with the bone graft material. Preferably the bone graft material of the invention is for use in the treatment of a subject.

The term “cell material” as used in context of the present invention shall in particular pertain to materials comprising biological cell material, such as living or dead cells, preferably living cells, as well as any secreted factors derived from the culturing of such cells. In certain preferred embodiments, however, the present invention pertains to cell material comprising living biological cells. The expression “biological cell” in context of the invention shall preferably refer to a mammalian cell, most preferably a human cell. Certain embodiments of the invention pertain to mesenchymal stromal cells as preferred biological cells in context of the invention. This may in some embodiments exclude other cells that can be derived from bone marrow fractions, such as for example hematopoietic progenitor cells, or erythropoietic progenitor cells. In the present disclosure, the term “mesenchymal stromal cell” is a multipotent stromal cell that can differentiate into a variety of cell types, including, but not limited to osteoblasts (bone cells), chondrocytes (cartilage cells), myocytes (muscle cells) and adipocytes (fat cells). These cells are also known as “mesenchymal stem cells” due to their multi-potency. This biologically important cell population is able to support hematopoiesis, can differentiate along mesenchymal and non-mesenchymal lineages in vitro, is capable of suppressing alloresponses and appears to be non-immunogenic. MSC are, in particular in context of the present invention, useful due to their regenerative function by paracrine or endocrine mechanisms, their immune-suppressive activity as well as their ability to form direct cell-to-cell interactions. These cells are known to either secrete certain proteins and/or compounds, which is turn influence the microenvironment around the cells, or when not directly expressing the proteins themselves, mesenchymal stromal cells are known to influence the downstream expression of other proteins, which for example, may be affected by the presence or absence of a mesenchymal stromal cell. That is to say that the presence of a mesenchymal stromal cell within the environment of another cell can cause the other cell to start producing, or even secreting, certain proteins. Therefore, disclosed herein are both compounds and/or proteins secreted by the mesenchymal stromal cells themselves and are therefore considered to be derived from MSCs as they are specifically obtained from MSCs, as well as proteins and compounds that are produced and/or secreted as a result of the presence of mesenchymal stromal cells within the cell environment. Included therein is also the downstream expression of proteins that is initiated via one or more expression cascades due to the presence of mesenchymal stromal cells in the microenvironment. MSC according to the invention may be obtained from a variety of sources such as bone marrow, adipose tissue, umbilical cord blood and others.

The MSC according to the invention is in some preferred embodiments provided as a monogenic composition, therefore, such composition comprises preferably MSC of only one genetic background (or derived from one single donor subject). Other embodiments, which might be preferred in context of the invention, then pertain to a MSC preparation comprising MSC derived from multiple genetically distinct donor subjects. Preferred MSC preparations are those which are prepared according to the methods described in PCT/EP2015/066083 (published as WO 2016/008895; disclosed herein by reference in its entirety, and specifically the method for generating MSC preparations comprising a step of pooling MSC from more than one genetically distinct donor before MSC isolation from such cell pool). The number of donors providing the samples to be pooled for later MSC isolation is preferably more than one preferably from at least 3, 4, 5, 6, 7, 8, 9 or 10 or more genetically distinct donor subjects. Preferably, MSC according to the present invention are obtained from bone marrow samples, such as bone marrow mononuclear cell (BMNC) fractions.

Hence, in certain preferred embodiments of the invention the cell material is allogenic to the subject treated with the bone graft material of the invention. As used herein, the term “allogenic” or “allogeneic” refers to cells, tissues or organs that are not genetically identical or are derived from a non-genetically identical source to the transplant recipient (e.g., a non-related donor), especially with respect to antigens or immunological reactions. Such cells, tissues or organs are called allografts, allogeneic transplants, homografts or allotransplants.

In certain preferred embodiments the invention in addition pertains to bone graft material that comprises scaffold material that is suitable for implantation into or attachment onto a mammalian bone. The term “scaffold material” is intended to refer to a composition that is capable of forming a scaffold, i.e. a pre-scaffold material. For example the scaffold material may comprise a composition that is capable of setting into a scaffold. The scaffold material itself may or may not have a structure of a scaffold until the scaffold material has formed the scaffold according to the methods herein. Reference to “a composition that is capable of forming a scaffold” may include the capability to form a scaffold with no further intervention/process steps or components. In an alternative embodiment, reference to “a composition that is capable of forming a scaffold” may include the capability to form a scaffold following further intervention/process steps according to the invention herein and/or following addition of components according to the invention herein. The term “scaffold” (may be interchanged with the term “matrix”) is understood to mean a solid mass of material having a 3-dimensional structure, which may for example be suitable to support cells. In embodiments of the invention, the scaffold may be porous, having interconnected pores or gaps.

In certain embodiments of the invention, the scaffold material is synthetic or natural, preferably wherein the scaffold material comprises calcium phosphate, preferably α-, or β-tricalciumphosphate (TCP), or comprises polylactic acid or polycarpolactone (or other biodegradable polymer), or a mixture of these materials. Natural scaffold material is for example obtained from bones or dental material. Naturally-derived bone scaffold materials are usually prepared by acid extraction of most of the mineralized component to result in so called demineralized bone matrix (DBM). Examples for naturally-derived materials are Bio-Oss® of the mineral portion of bovine bone or Algipore® a porous calcium phosphate material of algae. Autologous (or allogenic) bone is an ideal source of graft material, not only due to its biocompatibility, but also because natural bone grafts facilitate reossification of the defect site by promoting or conducting ingrowth of the patient's own bone tissue to the defect site. Methods for obtaining such material are well known to the person of skill in the art. An additional preferred source of natural bone scaffold material is the use of heat-inactivated spongiosa which is prepared by removing cartilage from a femoral head and heating it to more than 60° C., preferably to more than 80° C., preferably about 90° C., for more than 10 minutes (preferably about an hour).

The aforementioned β-TCP is one crystal form of a composition represented by Ca₃(PO₄)₂, which is a biocompatible compound that is stable at normal temperature. As the β-TCP, commercially available β-TCP powders and β-TCP blocks, for example, β-TCP blocks to be used as scaffold material may be used, or the β-TCP can be produced by a publicly known method (Japanese unexamined Patent Application Publication No. 2004-26648, etc.) or from an α-TCP powder (Japanese unexamined Patent Application Publication No. 2006-89311, etc.). Also, while the β-TCP composition of the present invention may be a β-TCP composition substantially composed of β-TCP or a β-TCP composition produced by mixing in a surfactant, water and so on, the β-TCP composition may be a β-TCP powder obtained by processing a β-TCP block into particles or granules, and besides that, the β-TCP composition is preferably a polycrystalline, high-strength and high-density β-TCP dense body having a low porous rate obtained by densificating a β-TCP powder by tablet pressing or after defoaming by sintering, and among such β-TCP dense bodies, one having a porous rate of 50% or more is preferable. In the present invention, a β-TCP dense body refers to β-TCP having a porous rate of 50% or more. Also in the present invention, powders, particles, and granules all indicate the granular form, and there is no substantial distinction among them. Pores in the β-TCP dense body are determined according to the size and number of spaces between the particles of the polycrystal.

The porous rate of β-TCP can be measured by using a micro CT (Bertoldi S, Farè S, Tanzi M C. Assessment of scaffold porosity: the new route of micro-CT. J Appl Biomater Biomech. 2011 September-December; 9(3):165-75. doi: 10.5301/JABB.2011.8863. Review) a mercury intrusion method, in which pressure is applied to force mercury into a pore and the specific surface area and pore distribution are obtained from the pressure applied and the amount of mercury intruded, or from the density using a calibration curve. It is also possible to measure the true density ρ of a β-TCP stone having a porous rate of 0% (a β-TCP sintered body having the same composition as the sintered body of an object to be measured but having no pores in the crystalline form) in advance, and from an apparent density ρ′ calculated from the volume and weight of the sintered body of an object to be measured, calculate the porous rate P(%) as P(%)=(1−ρ′/ρ)×100. Further, the porous rate P can also be measured using, for example, the ACCUPYC 1330 series (the product of Shimadzu Corporation). The porous rate in the β-TCP block as measured by the mercury intrusion method is preferably 50 to 90%, more preferably 60 to 85%, and even more preferably 70 to 80%. A β-TCP powder having a particle diameter of 75 to 105 μm can be produced by pulverizing a β-TCP block, and the porous rate of each powder thus produced as measured by the mercury intrusion method is preferably 50 to 90%, more preferably 55 to 85%, and even more preferably 57 to 80%, and a preferable average porous rate is 60 to 70%. Also, a β-TCP microgranule having a particle diameter of 25 μm or less can also be produced by pulverizing a β-TCP block into particles. The porous rate of the β-TCP microgranule thus produced as measured by the mercury intrusion method is preferably 50% or more, more preferably 50% to 90%. Examples of the β-TCP dense body can include a β-TCP powder, particle, granule, tablet, and columnar body having a porous rate of 50% or more. These β-TCP dense bodies may consist exclusively of β-TCP or comprise β-TCP as scaffold for the MSC seeded on the material as active ingredient, as well as other components.

For example, densification of a β-TCP powder by sintering is performed by sintering preferably at above 600° C. to 2000° C., more preferably at 750 to 1500° C., and even more preferably at 900 to 1300° C., and preferably for 1 to 100 hours, more preferably for 10 to 80 hours, and even more preferably for 20 to 50 hours. Further, sintering can be divided into multiple operations and performed at a plurality of different temperatures. For example, a rod-shaped β-TCP dense body can also be produced by the following method. A slurry is prepared by mixing calcium hydrogen phosphate, calcium carbonate, and water at an appropriate ratio, and the slurry thus prepared is allowed to undergo reaction while grinding, followed by drying. Subsequently, the solid material obtained after drying is pulverized and calcined to give a β-TCP powder. The β-TCP powder thus obtained is formed into a columnar body by compression forming. The resulting compression-formed columnar body is sintered at 600° C. to 1300° C. for about 20 hours.

In some embodiments, which might be preferred, the scaffold material is a nanoporous material. As used herein, the term “nanoporous” generally refers to a porous material (i.e. a calcium phosphate ceramic) whose average pore diameter is in the nanometer range (typically between 1 to 1000 nm). More preferably, the pores of the nanoporous material of the invention are communicating nanopores, which preferably are pores which are interconnected via channels. Such communicating pores preferably have a degree of porosity and interconnection that there is a channel connection from one side of the graft to the other. A porosity according to the invention is preferably a porosity of 50%-90%, preferably of 60%-70%; and/or wherein the nanopores have an average diameter of 50-600 μm, preferably of 100-500 μm; and/or wherein the nanopores are communicating nanopores.

In some further embodiments, the scaffold material is preferably not a decellularized tissue matrix obtained from natural, such a xenogeneic (non-human) tissues. The term “decellularized” or “decellularization” as used herein refers to a biostructure (e.g., a tissue or an organ, or part of an organ), from which the cellular content has been removed leaving behind an intact acellular infra-structure mainly composed of collagens.

In certain preferred embodiments of the invention the bone graft material is in the form of an implantable tablet, preferably a tablet having cylindrical shape (rod-shaped). The term “cylindrical shape” as used herein refers to any geometrical shape having the same cross section area throughout the length of the geometrical shape. The cross section of a cylinder within the meaning of the present invention may have any two dimensional shape, for example the cross section may be circular, oval, rectangular, triangular, angular or star shaped. In specific embodiments, the pharmaceutical compositions described herein have a cylindrical shape, wherein the end(s) are optionally tapered. In particular embodiments, a bone graft material according to the invention is a tablet having a height of 1 to 10 mm, preferably 3 to 5 mm, and/or a diameter of 1 to 15 mm, preferably 5 to 8 mm. In context of the herein disclosed treatment of bone disorders and injuries, the tablet of the invention with the herein disclosed dimensions showed surprisingly improved effects for the performance of the MSC during the treatment. As used herein, the “length” refers to the dimension of the longest axis of the tablet and the “height” refers to the dimension of the longest axis in the largest plane of the tablet. The diameter therefore refers to the axis perpendicular to the height.

In certain particular embodiments the bone graft material of the invention is cryopreserved, and thus optionally requires thawing immediately before surgical insertion or attachment to the bone of a patient. The term “cryopreservation”, as used herein, refers to the process of cooling and storing biological cells, tissues, or organs at low temperatures to maintain their viability. As a non-limiting example, cryopreservation can be the technology of cooling and storing cells at a temperature below the freezing point (e.g., −20° C. or colder, −80° C. or colder, or the like) that permits high rates of survivability of the cells upon thawing. In context of the invention the biological material seeded onto the scaffold material is ideally cryopreserved in order to provide an off-the-shelf product for implantation.

In a second aspect, the invention pertains to bone graft implant, comprising a scaffold material and a biological cell material, wherein the bone graft implant is in the form of an implantable tablet having a height of 1 to 10 mm, preferably 3 to 5 mm, and/or a diameter of 1 to 15 mm, preferably 5 to 8 mm.

In some embodiments, the bone graft implants may be made from allograft, autograft, xenograft, cortical, cancellous, cartilage, and combinations of, synthetic HA, B-TCP, Ceramic, PLA, PLGA, PLLA or other such materials and may be incorporated with growth factors, plasma (e.g., platelet rich plasma), platelets (e.g., intact platelets), cells, peptides or other such materials.

In some embodiments, the bone graft implants are cylinders, rectangular, trapezoidal, flat, convex, concave, and may have a channel on one or both sides for easy placement against a patient's bone or for buttressing against a bone. In some embodiments, the bone graft implants may be trimmed to size and snapped onto a rod by press fit or by means of sutures.

Preferably, the bone graft implant biological cell material comprises living cells, preferably allogenic or autologous. More preferably the biological cell material is the biological cell material as described herein for the first aspect of the invention.

In further embodiments the bone graft material, cellular composition or other compounds, materials and composition described herein in the various aspects of the invention preferably do not comprise added, and preferably immobilized, bone growth factors or cytokines. Such growth factors and cytokines are often added in order to render cells in bone graft material osteo-inductive. However, the materials of the present invention in all aspects and embodiments thereof preferably do not require such addition of osteo-inductive cytokines or growth factors since the cellular compositions of the invention are inherently osteo-inductive. Such cytokines include bone morphogenic protein (BMP), Indian hedgehog (IHH), transforming growth factor β (TGF3); bone morphogenetic proteins (BMPs); fibroblast growth factors (FGFs); Wnt ligands and β-catenin; insulin-growth factors (IGFs); collagen-1; Runx2; osteopontin; osterix; vascular endothelial growth factor (VEGF); platelet derived growth factor (PDGF); osteoprotegerin (OPG); NEL-like protein 1 (NELL-1); or a mixture thereof.

The bone graft implant according to the invention is preferably cryopreserved.

In a third aspect, the invention pertains to a method for producing or manufacturing a cryopreserved bone graft material, comprising the steps of
Seeding biological cell material as recited in any one of the aspects of the invention on a scaffold material as recited in any of the aspects of the invention to obtain a bone graft material, and
Immediately cryopreserving the bone graft material obtained in the first step.

Since freezing a biological sample containing living material such as cells bears the risk of sheering and destruction of the cell material, cryopreservation medium is used in order to minimize the loss of cell material during the process. As used herein, the term “cryopreservation medium” or “cryoprotectant” is a substance that is used to protect eukaryotic cells (and also tissues, organs) from freezing damage. Further, the cryopreservation agent or cryoprotectant may protect the eukaryotic cells (and also tissues or organs) from cold and heat shock, dehydration, and cryo-toxicity during cryopreservation. The cryopreservation agent or cryoprotectant may be cell penetrating or non-penetrating. Non-limiting examples of cryoprotectants include glycerol, DMSO (dimethyl sulfoxide), propylene glycol, ethylene glycol, acetamide, and methanol. However it should be cautioned that cryoprotectants have some disadvantages in that they can induce protein denaturation at higher temperature and cause cryoprotectant toxicity in cellular systems (like tissues and organs). The toxicity of the cryoprotectants is a major limitation to successful cryopreservation of eukaryotic cells. DSMO is a preferred cryoprotectant for use in accordance with the present invention.

Preferably, in some embodiments of the invention the cryopreservation is performed at −15° C. or less, preferably is −20° C. or less, preferably less than −50° C. (about −70° C. or −80° C.); preferably the cryopreservation comprises a step of freezing at a freezing temperature of less than −20° C., such as −40° C. or less, and subsequent storing of at temperatures higher than the freezing temperature.

In accordance with the present invention the scaffold material for us in the process of the invention is in granulate form, preferably wherein the granules have an average size of 0.1 to 3 mm, preferably 0.5 to 3 mm, more preferably 0.7 to 2.8 mm, more preferably 1.4 to 2.8 mm. However, in other embodiments the scaffold material is already provided in the form of an implantable body, such as a tablet or rod, preferably in cylindrical shape.

The biological cell material is seeded on the scaffold material in accordance with some embodiments of the invention in a density of at least 0.1×10⁶ cells per mL scaffold material, preferably of at least 0.5×10⁶ cells per mL scaffold material, more preferably of about 1×10⁶ cells per mL scaffold material.

In a fourth aspect, the invention pertains to a method of producing a bone graft implant, the method comprising the step of compressing a bone graft material recited in any one of the preceding claims into a tablet, preferably having a height of 1 to 10 mm, preferably 3 to 5 mm, and/or a diameter of 1 to 15 mm, preferably 5 to 8 mm.

In a fifth aspect, the invention pertains to a therapeutic kit, comprising
A first container comprising a scaffold material recited in any one of the preceding aspects,
A second container comprising a biological cell material recited in any one of the preceding aspects.

In some embodiments the present invention further provides a therapeutic kit comprising separate containers of the biological cell material and the scaffold material. In this aspect it is intended to mix the cell material directly with the scaffold material before medical use. For example, either the biological cell material is immediately combined with the scaffold material before surgical implantation, or alternatively, before the combination the biological cell material is expanded by in-vitro culturing to provide higher cell numbers before application.

The first and the second container may have any form suitable for packaging their respective materials. In some embodiments it might be however preferred that the container are formed such to allow a direct mixing of the content of the first container with the content of the second container to obtain the final product for use in the treatment. Syringes are one example of such containers.

The therapeutic kit of the invention may comprise additional components useful for realizing a medical treatment of the invention. Buffers, media, or instructions for use are non-limiting examples of such additional components.

In a sixth aspect, the invention pertains to a mesenchymal stromal cell (MSC), or a MSC preparation for use in the treatment of a disease, wherein the treatment comprises the administration of the MSC, or the MSC preparation, to a subject in need of the treatment by adhering the MSC or MSC preparation to a bone graft material to obtain an MSC-bone graft material, and implanting or attaching the MSC-bone graft material into/to one or more bone(s) of the patient.

In a seventh aspect, the invention pertains to a use of a MSC, or a MSC preparation, in the manufacture of a medicament, preferably a bone graft material, for use in the treatment of a bone injury or disorder. Such bone injuries may be selected from aseptic bone necrosis (spontaneous or therapy induced), bone fractures, fractures with secondary dislocation, pseudo-arthrosis, and reduced and delayed bone build-up. Bone disorder treatable by the invention are any condition affecting the development and/or the structure of the skeletal system. Bone disorders can include any disorder that results in an imbalance in the ratio of bone formation to bone resorption such that, if unmodified, a subject will exhibit less bone than desirable, or a subject's bones will be less intact and coherent than desired. Bone disorders may refer to disorders, diseases, conditions or traumas of bone, such as fractures, osteoporosis, osteoarthritis, or cancers. For example, a bone disorder may result from fracture, from surgical intervention or from dental or periodontal disease. These diseases have a variety of causes and symptoms known in the art. Bone disorders can affect people of all ages, from newborns to older adults. However, certain age groups are more likely to have certain types of disorders.

The uses and methods according to the above aspects may in certain preferred embodiments comprise a step of culturing the MSC, or MSC preparation and in particular the MSC comprised in the graft material, before implantation. Hence, in a preferred embodiment the uses and methods of the invention comprise a step of culturing for at least 6 h, preferably at least 12 h, more preferably at least 18 h, more preferably at least 24 h, even more preferably at least 36 h and most preferably at least 48 h or more. The indicated culture times are commenced immediately after thawing the frozen MSC preparation and before implantation. Maximum culture times are up to 96 h, or less, for example 7h—most preferred is a culturing time of 24 to 72 h, or for about 48 h.

Hence, there is furthermore provided a method for thawing a bone graft material according to the invention and preparing the bone graft material for implantation, the method comprising a step of culturing the MSC comprised in the bone graft material for at least 6 h, preferably at least 12 h, more preferably at least 18 h, more preferably at least 24 h, even more preferably at least 36 h and most preferably at least 48 h or more. The indicated culture times are commenced immediately after thawing the frozen MSC preparation and before implantation. Maximum culture times are up to 96 h, or less, for example 7h—most preferred is a culturing time of 24 to 72 h, or for about 48 h.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

In view of the above, it will be appreciated that the present invention also relates to the following itemised embodiments:

Item 1: A bone graft material for use in the treatment of a subject, comprising a scaffold material and a biological cell material, wherein the biological cell material comprises cell material genetically allogenic to the subject.
Item 2: The bone graft material for use according to item 1, wherein the biological cell material comprises living cells.
Item 3: The bone graft material for use according to item 1 or 2, wherein the biological cell material, and preferably the graft material is cryopreserved.
Item 4: The bone graft material for use according to any one of items 1 to 3, wherein the biological cell material comprises mesenchymal stromal cells (MSC) derived from any source, or comprises bone marrow mononuclear cells (BMC).
Item 5: The bone graft material for use according to any one of items 1 to 4, wherein the biological cell material comprises living cells of one donor subject, preferably of more than two genetically distinct donor subjects, preferably from at least 3, 4, 5, 6, 7, 8. 9 or 10 or more genetically distinct donor subjects.
Item 6: The bone graft material for use according to item 5, wherein the biological cell material is obtained from, or derived from, a pooled preparation of bone marrow samples, or bone marrow mononuclear cell fractions, of the genetically distinct donors, in accordance with a method as described in WO 2016/008895.
Item 7: The bone graft material for use according to any one of items 1 to 6, wherein the treatment comprises surgical insertion or attachment of the bone replacement material to a bone of the subject.
Item 8: The bone graft material for use according to any one of items 1 to 7, wherein the treatment is for promoting bone growth or healing, such as for example to treat a bone injury or disorder selected from bone fracture, bone trauma, arthrodesis, a bone deficit condition associated with post-traumatic bone surgery, post-prosthetic joint surgery, post-plastic bone surgery, post-dental surgery, bone chemotherapy treatment, congenital bone loss, post traumatic bone loss, post-surgical bone loss, post infectious bone loss, allograft incorporation or bone radiotherapy treatment, and preferably in the treatment of osteonecrosis (avascular necrosis (AVN)).
Item 9: The bone graft material for use according to item 8, wherein the osteonecrosis is a secondary disease caused by corticosteroid treatment, trauma, alcohol, sickle cell disease, leukaemia or other known causes of osteonecrosis, or is idiopathic osteonecrosis.
Item 10: The bone graft material for use according to any one of items 1 to 9, wherein the biological cell material is adsorbed on the scaffold material.
Item 11: The bone graft material for use according to any one of items 1 to 10, wherein the scaffold material is synthetic or natural, preferably wherein the scaffold material comprises calcium phosphate, preferably α- or β-tricalciumphosphate (TCP), or comprises polylactic acid or polycaprolactone (or other biodegradable polymer), or a mixture of these materials.
Item 12: The bone graft material for use according to any one of items 1 to 11, wherein the scaffold material is a nanoporous material with a porosity of 50%-90%, preferably of 60%-70%; and/or wherein the nanopores have an average diameter of 50-600 μm, preferably of 100-500 μm; and/or wherein the nanopores are communicating nanopores.
Item 13: The bone graft material for use according to any one of items 1 to 12, which is in the form of an implantable tablet, preferably a tablet having cylindrical shape.
Item 14: The bone graft material for use according to item 13, wherein the tablet has a height of 1 to 10 mm, preferably 3 to 5 mm, and/or a diameter of 1 to 15 mm, preferably 5 to 8 mm.
Item 15: The bone graft material for use according to any one of items 1 to 14, which is cryopreserved and requires thawing immediately before surgical insertion or attachment.
Item 16: A bone graft implant, comprising a scaffold material and a biological cell material, wherein the bone graft implant is in the form of an implantable tablet having a height of 1 to 10 mm, preferably 3 to 5 mm, and/or a diameter of 1 to 15 mm, preferably 5 to 8 mm.
Item 17: The bone graft implant according to item 16, wherein the biological cell material comprises living cells, preferably allogenic or autologous.
Item 18: The bone graft implant according to item 16 or 17, which is cryopreserved.
Item 19: The bone graft implant according to any one of items 16 to 18, wherein the biological cell material comprises mesenchymal stromal cells (MSC) derived from any source, or comprises bone marrow mononuclear cells (BMC).
Item 20: The bone graft implant according to any one of items 16 to 19, wherein the biological cell material comprises living cells of more than two genetically distinct donor subjects, preferably from at least 3, 4, 5, 6, 7, 8. 9 or 10 or more genetically distinct donor subjects.
Item 21: The bone graft implant according to any one of items 16 to 20, wherein the scaffold material is the scaffold material recited in any one of items 1 to 15.
Item 22: The bone graft implant according to any one of items 16 to 21, for a use recited in any one of items 1 to 15.
Item 23: A method for producing cryopreserved bone graft material, comprising the steps of
(i) Seeding biological cell material as recited in any one of items 1 to 22 on a scaffold material as recited in any one of items 1 to 22 to obtain a bone graft material, and
(ii) Immediately cryopreserving the bone graft material obtained in (a).
   Item 24: The method according to item 23, wherein the bone graft material is cryopreserved in a cryopreservation medium, for example a DSMO containing cryopreservation medium.
   Item 25: The method according to item 23 or 24, wherein cryopreservation is at less than −15° C. or less, preferably is −20° C. or less, preferably less than −50° C. (about −70° C. or −80° C.); preferably the cryopreservation comprises a step of freezing at a freezing temperature of less than −20° C., such as −40° C. or less, and subsequent storing of at temperatures higher than the freezing temperature.
   Item 26: The method according to any one of items 23 to 25, wherein the scaffold material is in granulate form, preferably wherein the granules have an average size of 0.1 to 3 mm, preferably 0.5 to 3 mm, more preferably 0.7 to 2.8 mm, more preferably 1.4 to 2.8 mm.
   Item 27: The method according to any one of items 23 to 26, wherein the biological cell material is seeded on the scaffold material in a density of at least 0.1×10⁶ cells per mL scaffold material, preferably of at least 0.5×10⁶ cells per mL scaffold material, more preferably of about 1×10⁶ cells per mL scaffold material.
   Item 28: A method of producing a bone graft implant, the method comprising the step of compressing a bone graft material recited in any one of the preceding items into a tablet, preferably having a height of 1 to 10 mm, preferably 3 to 5 mm, and/or a diameter of 1 to 15 mm, preferably 5 to 8 mm.
   Item 29: A therapeutic kit, comprising
(i) A first container comprising a scaffold material recited in any one of the preceding items,
(ii) A second container comprising a biological cell material recited in any one of the preceding items;
   wherein the (i) and (ii) are provided in the therapeutic kit in separate containers for combining immediately before surgical insertion.
   Item 30: The therapeutic kit according to item 29, further comprising two syringes suitable for combining the biological cell material with the scaffold material.
   Item 31: The therapeutic kit according to item 29 or 30, for use in medicine, preferably for use in the treatment of a bone disease or injury, and preferably of osteonecrosis.
   Item 32: A mesenchymal stromal cell (MSC), or a MSC preparation, for use in the treatment of a disease, wherein the treatment comprises the administration of the MSC, or the MSC preparation, to a subject in need of the treatment by adhering the MSC or MSC preparation to a bone graft material to obtain an MSC-bone graft material, and implanting or attaching the MSC-bone graft material into/to one or more bone(s) of the patient.
   Item 33: A use of a MSC, or a MSC preparation, in the manufacture of a medicament, preferably a bone graft material, for use in the treatment of a bone injury or disorder.

### BRIEF DESCRIPTION OF THE FIGURES

The figures show:

FIG. 1: shows the metabolic activity of MSC seeded on β-TCP, frozen and thawed (second bars) and of MSC cultured on β-TCP permanently at 37° C. (first bars). Mean values and standard deviation are shown. MSC were from the same donor, measurement was done in triplicate. Metabolic activity was assessed by means of MTT assay.

FIG. 2: shows a photograph of the tablet of the invention

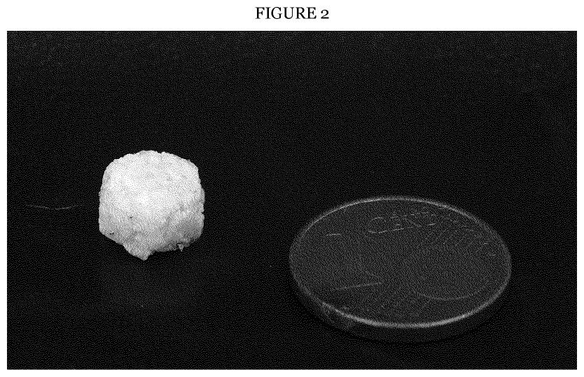

FIG. 3: shows human MSC (cell pool of n=8 donors) were placed for 3 h on β-TCP granules, which were modelled into the approximate dimensions of the planned MSC tablet (radius 4 mm, height 5 mm) by using a permeable form. MSC were applied at a density of 1×106 cells per cm³ β-TCP according to established seeding protocols and cultivated for 3 h at 37° C. and then first frozen at −80° C. (overnight) and then −196° C. (7 days) (experimental group). Metabolic activity was determined by MTT test in parallel cultures 24 h and 48 h after thawing. The control group consisted of MSC seeded at the same density and in the same manner on β-TCP, but incubated for 24 h at 37° C. only. Subsequently, the metabolic activity was also determined.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Comparative Example 1: Collection of Bone Marrow from 8 Healthy Third-Party Donors and Isolation of BM-MNCs

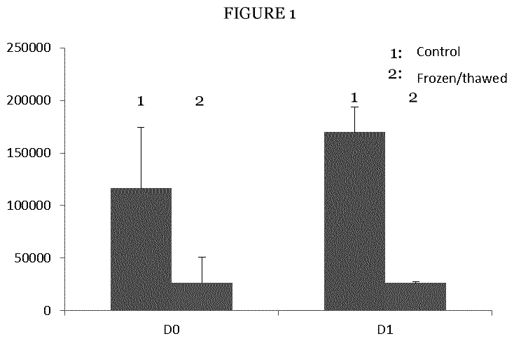

After obtaining a written informed consent, from each bone marrow donor were collected up to 250 ml additional bone marrow aspirate for the purpose of MSC banking with approval by the local Ethics Committee in full agreement with the Declaration of Helsinki. In total from 8 donors the inventors obtained 1.66 liters bone marrow. For isolation of bone marrow mono-nuclear cells by Ficoll-gradient the inventors used the Sepax machine as shown in FIG. 1. The absolute number of BM-MNCs per 1 ml of bone marrow after this isolation procedure was 3.3×10⁶±6.3×10⁵ cells. Total number of BM-MNCs which was obtained from eight donors after two washing steps was 9.86×10⁹. These cells were resuspended in cryomedium and distributed in the bags, that were frozen using a rate-controlled freezer and then stored in the vapour phase of liquid nitrogen until use.

### Comparative Example 2: Generation of Mesenchymal Stromal Cells from Bone Marrow Mononuclear Cells and Establishment of a MSC-Bank

To generate the MSC-Bank, bone marrow mononuclear cells from 8 donors were thawed, washed and pooled in DMEM supplemented with 5% PL. To find out the optimal concentration of platelet lysate for the adherence of progenitor cells of MSCs the inventors cultured BM-MNCs with both concentrations of PL: 5% and 10%. The obtained results have demonstrated that the 5% concentration of platelet lysate is much more efficacious in promotion of BM-MNCs and generation of MSCs than 10% concentration of PL. In addition, the inventors asked which of these two concentrations of PLs is better for clinical-scale expansion of MSCs. The inventors found that that the 10% concentration of PLs is significantly more efficient in expanding the MSCs than the 5% PL. Moreover, in both cases the unfiltered platelet lysates were more effective for generation and expansion of MSCs than the filtered ones. These preliminary experiments paved the way for establishing the master MSC-bank. Therefore, the inventors thawed the BM-MNCs from each donor and after washing twice they were pooled together and thereafter cultured for 14 days, as described in the section of methods. The inventors were able to generate from 9.89×10⁹ BM-MNCs 3.2×10⁸ MSCs of passage 1. These MSCs expressed the typical markers for MSCs, such as CD73, CD90 and CD105 but were negative for hematopoietic cell markers e.g. CD14, CD34, CD45. According to trypan blue staining the viability of these MSCs before freezing was 95±5%.

The total number of MSCs was distributed in 210 cryovials each containing 1.5×10⁶ MSC P1 and finally frozen in the gaseous phase of liquid nitrogen until use. The inventors referred to this set of vials as MSC-bank. The MSC as isolated according to comparative example 2 have improved allo suppressive potential as can be derived from WO 2016/008895.

### Example 1: Scaffold Materials

Dimensions and shape: Cylindrical, height 3-5 mm, diameter of 5-8 mm, depending on the size of the bone defect. A number of scaffolds can be combined to fill larger defects as they occur in the treatment of femoral head osteonecrosis.

The scaffold should consist of natural or synthetic matrices e.g. α-TCP, β-TCP, demineralized bone matrix, polylactic acid or polycaprolactone.

The scaffold should preferably be mechanically stable in order to prevent collapsing during surgical procedures and implantation. The scaffold should offer communicating macropores of the size range 100 μm to 500 μm. Pore sizes from 100 μm improve angiogenesis, while pore diameters of 300-400 μm have a positive effect on osteoconductivity. Micropores enabling surface enlargement, cellular adhesion and improvement of nutritional support should range from 1 μm to 10 μm. Overall porosity of the scaffold should be 60-70%, thus ensuring highest possible degree of porosity without compromising the mechanical strength.

The surface of the material should be rather smooth, rough microstructures in size range 1-10 μm should be prevented. Preferably, the surface consists of smooth slightly convex structures with a diameter ranging from 8-15 μm that are arranged in a honey comb similar matter.

### Example 2: Manufacture of a Bone Implant Graft

MSC derived from the cell bank of comparative example 2 were seeded to the scaffolds as described in Henrich et al, 2009, 2013, 2014; Seebach et al 2010, 2012, 2015. In brief, cells were harvested and preferably adjusted to a density of 1*10⁶ cells/mL (Range 1-1*10⁷ cells/mL). The cell suspension is carefully dripped on an equal volume of scaffold.

Non adsorbed cell suspension is carefully distributed once again evenly on the scaffold followed by 10 min incubation at 37° C., 5% CO2, 100% humidity. This procedure (Wetting of scaffold with non-adsorbed cell suspension followed by incubation) will be repeated two more times.

Such obtained functionalized graft material is ready for use or can be cryopreserved.

### Example 3: Cryopreservation of Bone Graft Implants

Cryopreservation will be performed preferably immediately after the seeding procedure but time span between seeding and cryopreservation may vary in a cell type dependent manner from 0 min to 24 hrs. In the latter case the scaffold will be stored in medium (DMEM+5% platelet lysate) at 37° C., 5% CO2, 100% humidity until cryopreservation procedure takes place.

The cell populated scaffolds were subjected to cryopreservation medium preferably consisting of physiologic NaCl solution (65% v/v, final NaCl concentration 0.7%), human serum albumin solution (HSA, 25% v/v, final HSA concentration 5%) and DMSO (10% v/v).

The cryopreservation medium is provided in sterile plastic bags. After addition of cell seeded scaffolds (range 1 to 20 scaffolds per bag), the bags are closed by heat sealing. The sealed bags were immediately subjected to a controlled rate freezer. Long term storage will be performed in liquid nitrogen vapor phase. The possibility for short term storage at −30° C. to −20° C. will be analysed.

### Example 4: Thawing and Use of Bone Graft Implants

Opening of sterile envelope and immediate subjection of the scaffold to a 50 mL vial filled with room temperature or prewarmed saline+HSA (0.5-5%, cleansing solution) followed by 5 min incubation in order to remove DMSO from the scaffold. The scaffold can be aseptically removed from the cleansing solution and should be placed immediately into the bone defect.

### Example 5: Medical Use of Bone Graft Implants

MSC were seeded on approximately 200 μL densely compressed β-TCP granules (size 1.4-2.8 mm) in a density of 1*10⁶ cells/mL β-TCP-scaffold. The seeding procedure consists of repeated dripping of the cell suspension over the scaffolds during a period of 30 minutes. Subsequently scaffolds were immediately frozen at −80° C. overnight in a medium containing 10% DMSO and 90% FCS. As control, MSC on β-TCP (same donor) were cultivated at 37° C. in parallel. All procedures described in the following were also performed with the control approach. Next day the well plate containing the scaffolds was placed for 3 min in a water bath (37° C.) in order to enable recovery of scaffolds. Scaffolds were then immediately dropped into 30 mL prewarmed (room temperature) medium (RPMI+10% FCS) for 1 min and recovered by filtering with cell strainer (100 μm mesh). Scaffolds were equally distributed into a 96-well plate using sterile forceps and 100 μL medium (Mesencult+supplements) were added to each well. In order to assess metabolic activity of the MSC on the scaffold an MTT assay was performed in triplicate following the instructions of the manufacturer. Incubation time with MTT reagent was 4 hrs. In order to assess long term survival of the MSC a portion of cells was cultured for additional 24 hrs after thawing followed by an MTT assay.

Metabolic cell equivalent (MCE) of frozen and thawed MSC was 2.7*10⁴ and around 25% of MSC cultured at 37° C. on β-TCP (5.8*104 MCE). If MSC were cultured additional 24 hrs after thawing, the metabolic activity remained approximately constant (2.6*10⁴ MCE) compared to cell activity directly measured after thawing (2.7*10⁴ MCE) whereas the metabolic activity of MSC cultured permanently at 37° C. increased further (1.7*10⁵ MCE) compared to the initial value (1.2*10⁵ MCE, FIG. 1). These results indicate that MSC seeded on a β-TCP scaffold can be stored frozen at −80° C. and remain in part vital. Furthermore, it can be concluded that frozen/thawed MSC did not further deteriorate 24 hrs after thawing, though they did not improve either. One might assume that the freeze/thaw procedure induced a lag phase of MSC proliferation.

### Example 6: Optimization of Cell Seeding as Well as Cryopreservation, Thawing and Reconstitution of the Cell-Populated Scaffolds

Human MSC-Pool (8 donors, passage 2) was obtained from the Clinic for Pediatrics and Adolescent Medicine. The cells were further cultivated up to a maximum of the 5th passage and used in experiments, or cryopreserved for later experiments. MSC phenotype was activated by FACS analysis (CD90+, CD105+, CD34−, CD45−) and the ability for osteogenic differentiation.

Using the MSC pool, our previous results were reproduced in a series of pilot experiments (n=3), which showed that hMSC, seeded on β-TCP scaffold and cultivated for a short time (3 h) at 37° C., are reduced metabolically active after freezing and thawing.

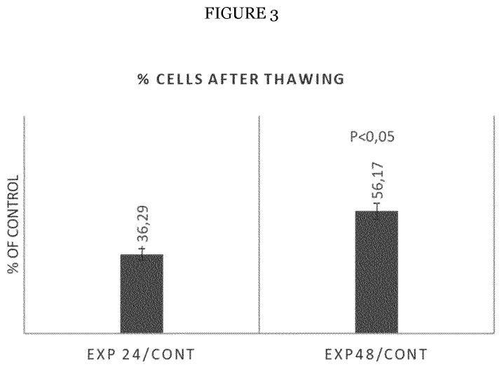

The metabolic activity of the cells was measured on the day after sowing for the control group and 1 and 2 days after rethawing using the MTT test. The mean metabolic activity of the cells of the experimental group increased significantly with increasing cultivation time after thawing (see FIG. 3: 36.3% of the control on day 1 and 56.2% of the control on day 2 after thawing; p<0.05, n=3).

1. A bone graft material for use in the treatment of a subject, comprising a scaffold material and a biological cell material, wherein the biological cell material comprises cell material genetically allogenic to the subject.

2. The bone graft material for use according to claim 1, wherein the biological cell material comprises mesenchymal stromal cells (MSC) derived from any source, or comprises bone marrow mononuclear cells (BMC).

3. The bone graft material for use according to claim 1 or 2, wherein the biological cell material comprises living cells of one donor subject, preferably of more than two genetically distinct donor subjects, preferably from at least 3, 4, 5, 6, 7, 8. 9 or 10 or more genetically distinct donor subjects.

4. The bone graft material for use according to any one of claims 1 to 3, wherein the treatment comprises surgical insertion or attachment of the bone replacement material to a bone of the subject.

5. The bone graft material for use according to any one of claims 1 to 4, wherein the treatment is for promoting bone growth or healing, such as for example to treat a bone injury or disorder selected from bone fracture, bone trauma, arthrodesis, a bone deficit condition associated with post-traumatic bone surgery, post-prosthetic joint surgery, post-plastic bone surgery, post-dental surgery, bone chemotherapy treatment, congenital bone loss, post traumatic bone loss, post-surgical bone loss, post infectious bone loss, allograft incorporation or bone radiotherapy treatment, and preferably in the treatment of osteonecrosis (avascular necrosis (AVN)).

6. The bone graft material for use according to claim 5, wherein the osteonecrosis is a secondary disease caused by corticosteroid treatment, trauma, alcohol, sickle cell disease, leukaemia, or is idiopathic osteonecrosis.

7. The bone graft material for use according to any one of claims 1 to 6, wherein the scaffold material is synthetic or natural, preferably wherein the scaffold material comprises calcium phosphate, preferably α- or β-tricalciumphosphate (TCP), or comprises polylactic acid or polycarpolactone (or other biodegradable polymer), or a mixture of these materials.

8. The bone graft material for use according to any one of claims 1 to 7, wherein the bone graft material is in the form of a tablet, and the tablet has a height of 1 to 10 mm, preferably 3 to 5 mm, and/or a diameter of 1 to 15 mm, preferably 5 to 8 mm.

9. A bone graft implant, comprising a scaffold material and a biological cell material, wherein the bone graft implant is in the form of an implantable tablet having a height of 1 to 10 mm, preferably 3 to 5 mm, and/or a diameter of 1 to 15 mm, preferably 5 to 8 mm.

10. The bone graft implant according to claim 10, wherein the biological cell material comprises mesenchymal stromal cells (MSC) derived from any source, or comprises bone marrow mononuclear cells (BMC).

11. The bone graft implant according to any one of claims 10 to 12, for a use recited in any one of claims 1 to 9.

12. A method for producing cryopreserved bone graft material, comprising the steps of
(i) Seeding biological cell material as recited in any one of claims 1 to 8 on a scaffold material as recited in any one of claims 1 to 8 to obtain a bone graft material, and
(ii) Immediately cryopreserving the bone graft material obtained in (a).

13. The method according to any one of claims 23 to 26, wherein the biological cell material is seeded on the scaffold material in a density of at least 0.1×10⁶ cells per mL scaffold material, preferably of at least 0.5×10⁶ cells per mL scaffold material, more preferably of about 1×10⁶ cells per mL scaffold material.

14. A therapeutic kit, comprising
(i) A first container comprising a scaffold material recited in any one of the preceding claims,
(ii) A second container comprising a biological cell material recited in any one of the preceding claims;
wherein the (i) and (ii) are provided in the therapeutic kit in separate containers for combining immediately before surgical insertion.

15. A mesenchymal stromal cell (MSC), or a MSC preparation, for use in the treatment of a disease, wherein the treatment comprises the administration of the MSC, or the MSC preparation, to a subject in need of the treatment by adhering the MSC or MSC preparation to a bone graft material to obtain an MSC-bone graft material, and implanting or attaching the MSC-bone graft material into/to one or more bone(s) of the patient.
